(19) United States
(12) Patent Application Publication
(10) Pub. No.: US 2011/0184353 A1
(43) Pub. Date: Jul. 28, 2011

(54) Subcutaneous injection port

(76) Inventor: Mary DeMaria, Farmington, CT (US)

(21) Appl. No.: 12/657,633

(22) Filed: Jan. 25, 2010

(51) Int. Cl. A61M 31/00

(52) U.S. Cl. 604/288.01

(57) ABSTRACT

An implantable surgical injection port includes a housing defining a fluid reservoir; a septum disposed across an opening of the housing so as to enclose the fluid reservoir and having at least a portion of which is penetrable by a needle; and a collar having an upper surface facing opposite the septum. The upper surface defines an exterior and an interior perimeter that circumscribes at least the needle penetrable portion of the septum. At least one of the interior perimeter and the exterior perimeter defines a shape that is not elliptical. In on-limiting embodiments, the shape is a heart, a star, or an animal. The shape that is not elliptical may have one or more angles of which a vertex faces inward or outward of the interior or exterior perimeter. In this manner a patient can personalize the look of their implanted injection port and not be embarrassed by it.

### TECHNICAL FIELD

The exemplary and non-limiting embodiments of this invention relate generally to subcutaneous injection ports, which form a portion of what is sometimes referred to as totally implantable venous access systems (TIVAS).

### BACKGROUND

Implantable injection ports are well known in the medical arts; see for example U.S. Pat. No. 4,701,159 and US Patent Application Publications 2001/0037094, 2005/0131325, 2006/0217673, and 2009/0221976. Such injection ports are implanted subcutaneously in a patient and with a catheter line they form a totally implantable venous access system TIVAS that is wholly implanted within a patient's body.

FIG. 1A illustrates an exemplary prior art TIVAS 10. The catheter 30 has a distal end 32 inserted into a vein of the patient, typically passing into the superior vena cava just upstream of the right atrium at the cavoatrial junction but also sometimes threaded through a cephalic vein, basilic vein or brachial vein enroute toward larger veins. A proximal end 31 of the catheter 30 attaches to a discharge stem 24 of the injection port 20. The injection port 20 has a cavity or reservoir 23 from which medicinal fluids flow into the patient's circulatory system via the catheter 30. Such fluids enter the cavity 23 by needle injection through a septum 22 of the port 20. Some ports retain position within a patient's body by use of protruding flanges 25 or loops which give a larger surface area to which growing tissue can attach and a greater moment arm for anchoring tissue to resist rotation of the port 20. Other ports are anchored via staples or stitches through pre-fabricated holes in the housing 21. The entire port 20 is surgically implanted within the patient's body, so any needle injecting medicinal fluid into the reservoir 23 first passes through the patient's skin which is stretched over the septum 22.

The advantage of an implanted port is most pronounced for patients undergoing long term injection therapy, such as for example chemotherapy patients, long-term antibiotic patients, and patients undergoing total parenteral nutrition. The skin overlying the septum becomes desensitized over time so that the patient either does not feel each needle stick or feels it much less than if there were an injection at a virgin site each time. The patient's body need heal only a skin deep puncture on each subsequent injection since the entire passageway from septum to vein is implanted only once. This also leads to a substantially reduced risk of infection after the TIVAS 10 is implanted.

Despite the copious benefits summarized above, some patients decline to have a TIVAS implanted because they are cosmetically objectionable. FIG. 1B is an image generally showing the cause of this concern. In order to be effective in desensitizing the overlying skin, the port is disposed directly under the fat and skin layers, very near the surface. This is one reason that ports are often located on the sternum or upper right chest as in FIG. 1B; the fatty layer at the abdomen is much thicker which can leave the port itself more prone to flipping or migrating in the patient's body. A deeply implanted port that is well anchored may still be difficult for a medical professional to find, resulting in needle sticks sometimes missing the septum.

Even if patients who initially decline to have a port implanted later agree that the implanted port is preferable to having virgin injection sites stuck with a needle each time routine medication is necessary, the end result is a period of time during which the patient is subjected to unnecessarily increased risk of infection (depending on what other method of injection was used), discomfort, and also emotional trauma due to the patient's concern for cosmetics. At least for chemotherapy patients, all this occurs at a time when the patient's energies need to be reserved for recovering from his/her ongoing treatment.

As shown at FIG. 1B, the upper chest region is a typical area at which these injection ports are disposed. It is more difficult for female patients to conceal with clothing the existence of an implanted port at this region since the cut of many common women's clothing is low enough to expose that area.

What is needed in the art is a way to alleviate or reduce the unsightliness of an implanted port so that more patients can exploit the above utilitarian benefits, or at least exploit those benefits at an earlier stage of their long-term medical treatment.

### SUMMARY

The foregoing and other problems are overcome, and other advantages are realized, by the use of the exemplary embodiments of this invention.

In a first aspect thereof the exemplary embodiments of this invention provide an implantable surgical injection port comprising a housing, a septum and a collar. The housing defines a fluid reservoir. At least a portion of the septum is penetrable by a needle and is disposed across an opening of the housing so as to enclose the fluid reservoir. The collar has an upper surface facing opposite the septum, and the upper surface defines an exterior perimeter and an interior perimeter that circumscribes at least the needle penetrable portion of the septum. At least one of the interior perimeter and the exterior perimeter defines a shape that is not elliptical.

In a particular embodiment, the interior perimeter comprises at least one angle having a vertex facing inward of the interior perimeter. In another particular embodiment, the exterior perimeter comprises at least one angle having a vertex facing outward of the interior perimeter. These two particular embodiments are combined in several of the illustrated examples which are detailed more fully below.

### BRIEF DESCRIPTION OF THE DRAWINGS

FIG. 1A is a simplified schematic diagram of a subcutaneous injection port with catheter according to the prior art.

FIG. 1B is a close-up image of a patient's upper chest area with an injection port implanted below the skin.

FIG. 2A is a plan view of an exemplary embodiment of the invention in which the collar overlying the septum of the injection port is in the shape of a heart.

FIGS. 2B-C are respective perspective and sectional views of the embodiment shown at FIG. 2A.

FIG. 3 is a plan view of a collar according to an embodiment of the invention in which the interior perimeter of the collar is circular and the exterior perimeter of the collar defines a five pointed star.

FIG. 4 is similar to FIG. 3 but the interior perimeter of the collar defines crossed ellipses and the exterior perimeter of the collar defines a four pointed star.

FIG. 5 is a plan view of a collar according to an embodiment of the invention in which the interior perimeter of the collar defines mostly an ellipse and the exterior perimeter of the collar defines a fish-shape.

FIG. 6 is a plan view of a collar according to an embodiment of the invention in which the exterior perimeter of the collar defines a turtle-shape.

FIG. 7 is an exploded perspective view of an embodiment of the invention having a secondary collar similar to that shown at FIG. 3 and which is removably coupled to a primary collar of the injection port to which the septum is attached.

FIGS. 8-9 are additional exemplary embodiments of the invention each having a collar with a circular interior perimeter and a non-elliptical exterior perimeter.

### DETAILED DESCRIPTION

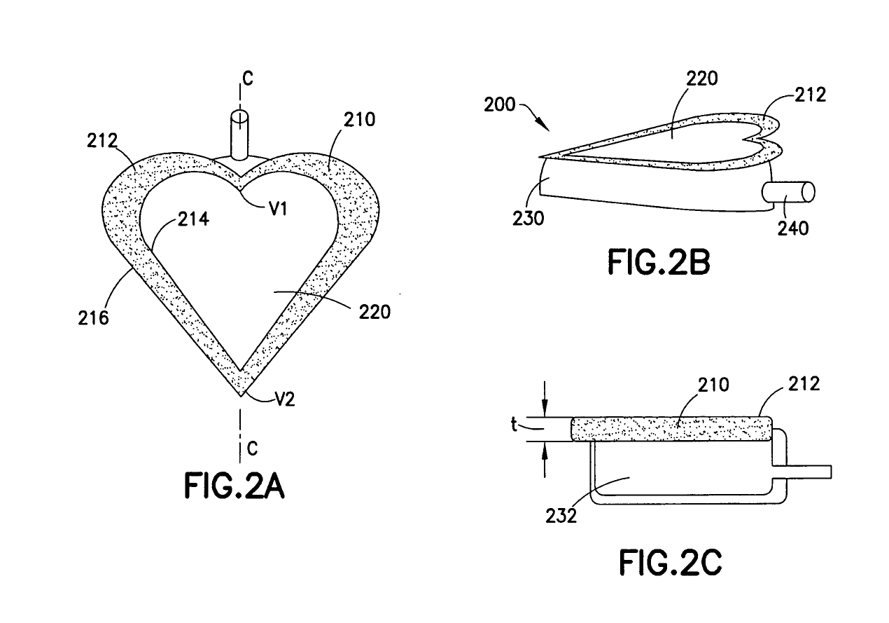

An exemplary but non-limiting embodiment of the invention is shown in plan view at FIG. 2A with perspective view at FIG. 2B and sectional view at FIG. 2C. FIG. 2C is taken along section line C-C of FIG. 2A. The implantable injection port 200 has a collar 210 that circumscribes a septum 220 and which is made to have a decorative shape at least at the collar surface 212 which faces and abuts the patient's skin. Typically this surface 212 will abut the fatty layer underlying the patient's skin once the injection port 200 is implanted. The collar 210 may be referred to as a tissue cuff, into which the fibrous tissue of the patient grows so as to anchor the injection port 200 against relative movement inside the patient's body. The various collars shown at FIGS. 2-9 are each offset by shading.

The injection port 200 includes a housing 230 that defines a fluid reservoir 232. In an embodiment the reservoir 232 defined only by the housing 230 has an open top. The septum 220, or at least a portion of it since it may be stretched partially over a sidewall of the housing 230, is disposed across that open top of the housing 230 so as to enclose the fluid reservoir 232. The injection port 200 also includes a discharge stem 240 which penetrates a sidewall of the housing 230 so as to define a passageway between the enclosed fluid reservoir 232 and exterior of the housing 230. A distal end of the stem 240 is particularly adapted to removably mate with a catheter line or tube.

The upper surface 212 of the collar 210 faces away from the septum 220, as is best shown at FIG. 2C. In an embodiment the collar overlies the septum and affixes the septum 220 to the housing 230, in which case a thickness t of the collar 210 as measured from the septum 220 to the upper surface 212 defines a distance by which the upper surface 212 is spaced from the septum 220. Since the upper surface 212 is not necessarily flat this distance may not be a singular value. As will be seen with respect to FIG. 7 this distance may be greater in certain embodiments than the thickness of the decorative collar 210, but in all instances the upper surface 212 of the collar 210 is raised above the plane in which the septum 220 lies.

By way of example and not limitation, the housing 230 is made of a plastic (polysulfone for example), surgical stainless steel or titanium. The housing can define a single reservoir as illustrated, or dual/multiple reservoirs as is known in the art with different stems penetrating the different reservoirs. In a preferred embodiment the housing 230 is circular- or elliptical-cylindrical in shape, about 25 mm in diameter and 15 mm in height (the direction of the thickness t at FIG. 2C) but other sizes are not excluded for other embodiments and the height of the housing 230 can vary widely, particularly for multi-reservoir embodiments. The housing 230 is made of a needle impenetrable material in the preferred embodiment. The stem 240 may also be made from any of the above housing materials.

The septum 220 is made of an elastomeric material that is penetrable by a hypodermic needle and that is also self-sealing after such needle penetration. Exemplary but non-limiting materials for the septum include silicone and silicone rubber. The collar 210 may also be made from any of the above septum materials, or also from a pliable and surgical grade plastic. Because the thickness t of the collar 220 is much greater than a thickness of the septum 220, the collar 210 will exhibit a much higher resistance to flex than the septum 210 even if both are made from the same material. As will be detailed below, a wire (metallic or non-metallic) or similar resilient member may be implanted within at least portions of the collar 210 to increase that resistance to flex. In an embodiment the thickness t of the collar 210 is greater than about 3 mm and preferably greater than 5 mm. In another embodiment the thickness t of the collar 210 is at least an order of magnitude greater than the septum 220 thickness.

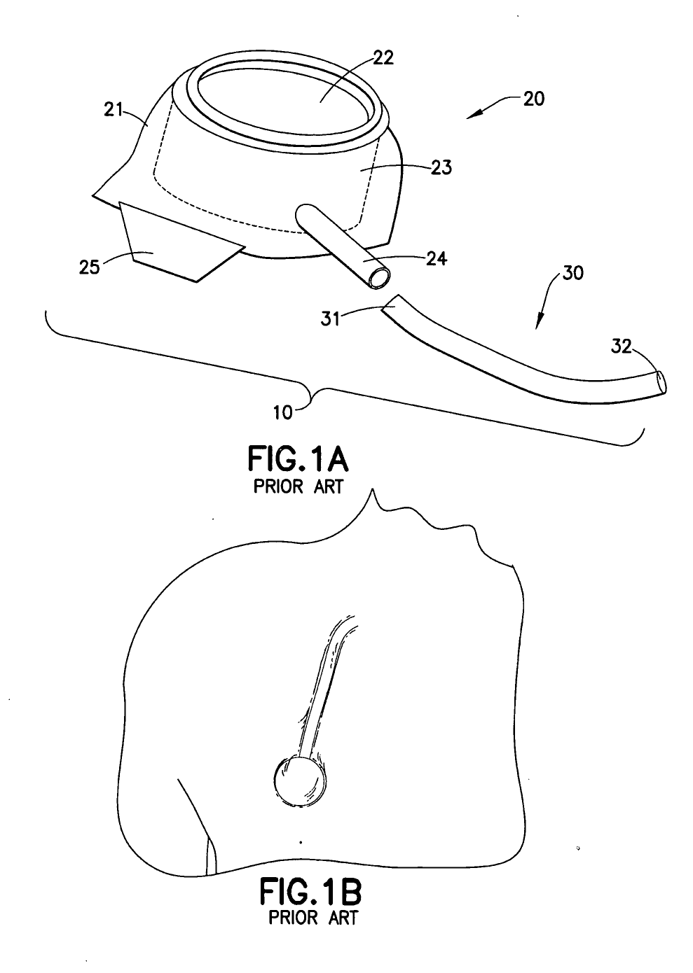

Conventional collars are circular in shape as shown at FIG. 1A. There is a utilitarian aspect to this in that a circle provides the largest area for injection while simultaneously providing the smallest perimeter to be implanted into the patient's body. The continuously arcuate exterior circumference of a conventional circular collar also eliminates angles over which the patient's skin would overlie, thereby avoiding the potential for puncture, reducing point stresses from outward facing angles, and providing a substructure over which the patient's skin can form a dome for more ready healing than other more irregular shapes. Since a circle is a special case of an ellipse in which the two ellipse foci are co-located, much of the above utilitarian benefits also apply, with only a marginal reduction in those benefits, if the collar were elliptical in shape.

Unlike such conventional circular collars, a collar according to exemplary embodiments of the present invention has an interior and an exterior perimeter, at least one of which defines a shape that is not elliptical. The embodiment of FIGS. 2A-C has the non-elliptical shape of a heart in both the interior perimeter 214 and the exterior perimeter 216. Another feature of the FIG. 2A-C embodiment is that for both the interior perimeter 214 of the collar 210 and the exterior perimeter 216 of the collar 210 there is an angle with its vertex V1 pointing inward of the interior and exterior perimeters 214/216 and another angle with its vertex V2 pointing outward of the interior and exterior perimeters 214/216. The angle with outward facing vertex V2 is acute as illustrated at FIG. 2A. While the inward facing angle has an obtuse vertex V1 in the heart-shaped collar 210 embodiment shown at FIG. 2A, in other embodiments of the heart-shaped collar 210 that same inward facing angle can define a vertex V1 that is an acute angle. As is conventional, the direction in which the vertex of an angle points is along the line that bisects that same angle.

When implanted subcutaneously in a patient, the upper surface 214 of the collar 210 defines a design that remains visible despite being implanted. Whereas the image at FIG. 1B shows a plain circle with the catheter line running upwards along the patient's chest, the heart-shaped collar 210 of FIGS. 2A-C is a more purposeful act on the part of the patient to show others his/her artistic expression. This outward artistic impression is not unlike an individual having a tattoo placed prominently on their body; often it is to display to others an expression of the tattoo bearer. As will be seen, there are many different shapes that can be formed by the collar according to embodiments of this invention, thereby rendering the outward appearance of the implanted port personalizable by the patient.

The end result is that chemotherapy patients and others undergoing long term injection medications are given a reason to embrace the concept of having an injection port implanted rather than resist it, because they can express their own personality a bit more than a generic circle of the prior art ports as in FIGS. 1A-B. Rather than silently endure confused stares from unknowing friends and strangers who see the outline of a prior art circular port just beneath the patient's chest, the patient can more confidently display the design they've chosen of their own accord. Rather than silently pretend the prominent but awkward prior art port does not exist, the injection port according to these teachings can become a positive conversation piece of which both an observer and the patient are more likely to openly discuss, first as to form and then perhaps as to function. In this manner, at least some additional patients who would otherwise resist implantation of a conventional port with circular collar will agree to the implantation of an injection port having its collar made into a design, particularly if the patient can select from among many designs.

FIG. 3 is a plan view of a collar 310 according to an embodiment of the invention in which the interior perimeter 314 of the collar 310 is circular and the exterior perimeter 316 of the collar 310 defines a five pointed star. The exterior perimeter 316 has five obtuse angles each having a vertex V1 pointing inward of the exterior perimeter 316, and further has five acute angles each having a vertex V2 pointing outward of the exterior perimeter 316.

The FIG. 3 embodiment further includes a wire 318 embedded within the material of the collar 310 and fully enveloped by that material to provide added rigidity to the arms of the star which extend furthest beyond the interior perimeter 314. This is because once the injection port 310 is implanted the patient's skin will be stretched over the entire collar 310, tending to flex down those portions extending furthest from the support of the underlying rigid housing 230. The embedded wires 318 or other distinct supporting members help assure those extending portions have sufficient rigidity so the design of the collar 310 shows through after implantation. The term wire is not to imply a metal member necessarily. Particularly for chemotherapy patients, a metal wire might interfere with routine MRI scans and so the wire may be a composite compound such as a stiff plastic or carbon. Fully encasing the wire, of whatever material, within the rubber-silicone material of the collar aids in isolating the wire material from biological contact with the patient's body.

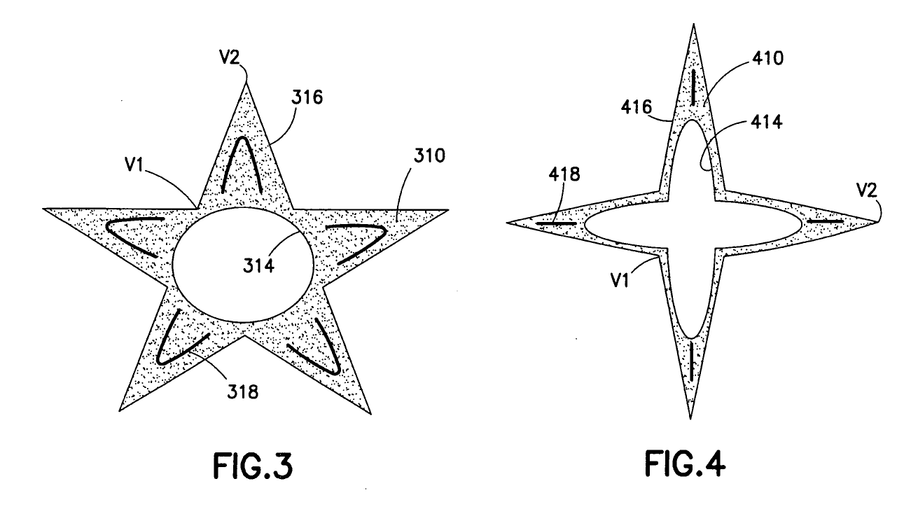

FIG. 4 is similar to FIG. 3 but the interior perimeter 414 of the collar 410 is in the shape of two crossed ellipses, and the exterior perimeter 416 of the collar defines a four pointed star. The exterior perimeter 416 has four obtuse angles each having a vertex V1 pointing inward of the exterior perimeter 416, and further has four acute angles each having a vertex V2 pointing outward of the exterior perimeter 416. Additionally, the interior perimeter 414 has four angles whose vertex points inward of the interior perimeter 414. The extending arms of the FIG. 4 star are also supported with wires 414 or other stiffening members.

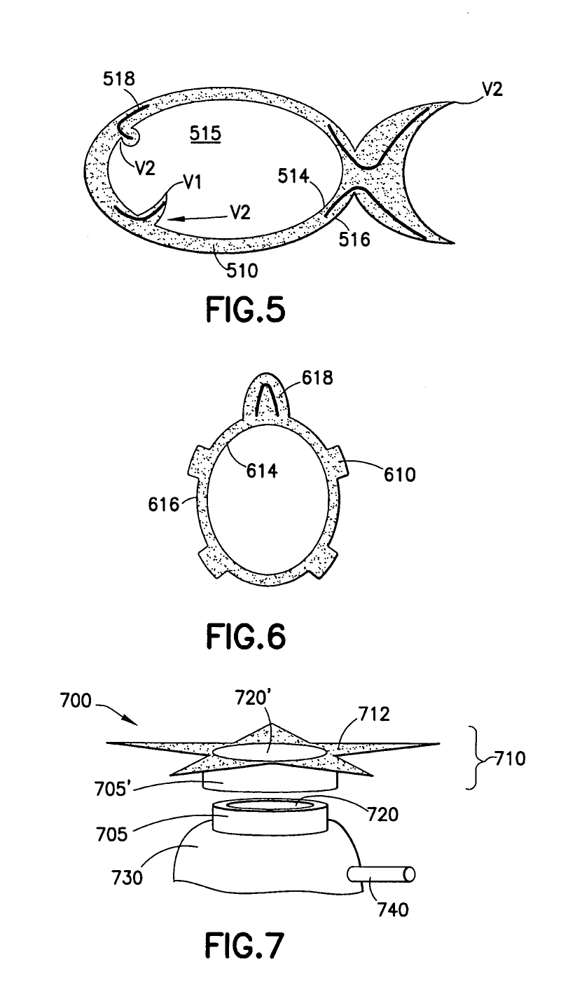

FIG. 5 is a plan view of a collar 510 according to an embodiment of the invention in which the interior perimeter 514 of the collar 510 defines mostly an ellipse, interrupted by interior protrusions 515 in the shape of a fish eye and a fish mouth. These are also supported by embedded wires 518 for added rigidity. These interior protrusions 515 diminish the area of the septum 520 that is available for a medical technician to inject a needle, but only marginally so while giving the advantage of greatly enhanced design options for the patient and manufacturer to choose from as is evident from FIG. 5. The exterior perimeter 516 of the collar 510 defines a fish-shape largely due to a crescent-shaped portion extending from the basic ellipse that forms a fish tail.

In the FIG. 5 embodiment, there are two angles at the tail fin which have vertexes V2 pointing outward of the exterior perimeter 516. There is also at the fish eye two angles along the interior perimeter 514 having vertexes V2 pointing outward of the interior perimeter 514, though these angles are not likely to be visible through the patient's skin once implanted and so a choice may be made to manufacture the eye with only arcuate lines. The fish mouth has a distinctive angle whose vertex V1 points inward of the interior perimeter 514 though, and would be visible once implanted. There is also an angle where the fish mouth meets the elliptical portion of the interior perimeter 514 whose vertex V2 points outward of the interior perimeter 514, which also would be visible once implanted.

In order to reduce point-stresses on the patient's skin after implantation, the acute inward facing angle of the fish mouth and the outward facing angles at the tail fin may have rounded vertexes without departing from the definition of being an angle.

FIG. 6 is a plan view of a collar 610 according to an embodiment of the invention in which the interior perimeter 614 defines an ellipse and the exterior perimeter 616 defines a turtle-shape. There is also an embedded wire 618 within the collar 610 to add rigidity to the head extension.

FIGS. 5-6 illustrate by example two animal shapes. The invention is readily adaptable to other animal shapes or to general design shapes such as those shown at FIGS. 2-4. For example and with respect to FIG. 6, extending the turtle ‘legs’ further and expanding the head into a more circular shape renders the design a teddy bear. The example stars of FIGS. 3-4 can be made with three points or six or more points. From these various embodiments it can be seen that the interior perimeter and the exterior perimeter may be the same shape such as at FIG. 2A or may be radically different shapes such as at FIG. 3. Where the fluid reservoir (232 of FIG. 2) is an elliptical-cylinder (of which a circular-cylinder is a special case), at least one of the interior perimeter and the exterior perimeter defines a shape that differs from the perimeter shape of the fluid reservoir.

FIG. 7 is an exploded perspective view of an embodiment of the invention having a secondary collar 710, which in this case defines a similar shape as the collar 310 shown at FIG. 3. But in the FIG. 7 embodiment the secondary collar 710 is removably coupled to a primary collar 705 disposed on the housing 730 of the overall injection port 700. The secondary collar 710 has the upper surface 712 which bears the design which will be visible through the patient's skin as detailed by example above. The secondary collar 710 is removably coupled to the housing 730 via a surface 705′ which mates to with a complementary surface of the primary collar 705. As shown the mating surfaces are cylinder surfaces defined by circular protrusions that fit one inside the other.

The advantage of this embodiment is manufacturing efficiency; the manufacturer need only make in quantity the lower portion having the housing 730, primary collar 705 and stem 740; and can manufacture more limited numbers of the secondary collar 710 but in a wider variety of designs so that the patient still has a selection of choices, while at the same time inventory management is simpler to enable those wider patient choices than if each choice was an entire port.

In the embodiment of FIG. 7, the septum 720′ may be affixed to the secondary collar 710 so that the reservoir is not enclosed until the secondary collar 710 is affixed to the primary collar 705, or more preferably the septum 720 is affixed to the housing 730 by the primary collar 705 itself. In the latter case, the port 700 may be implantable without the secondary collar 710 in which case the primary collar 705 would be in subcutaneous contact with the patient's skin/fatty tissue layers and would show through as a circle similar to that of FIG. 1B. In other embodiments the primary collar 705 is strictly a means to attach with the secondary collar 710 and the port 700 can only be implanted subcutaneously when the secondary collar 710 is included with it.

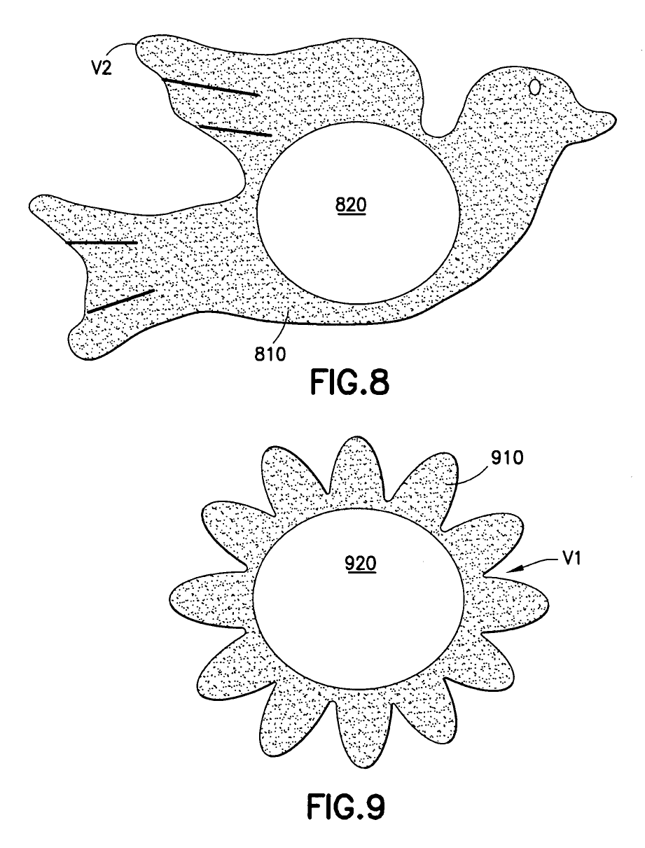

FIG. 8 illustrates another embodiment of the collar 810 in which the interior perimeter is circular and the exterior perimeter is in the shape of a flying dove. This particular embodiment has longer extensions away from the circular interior perimeter, demonstrating that the entire collar material can be a stiffer rubber-silicone compound than is conventional for the subcutaneous ports now in use such as that shown at FIGS. 1A-B. The exposed portion of the septum 820 is circular due to the circular interior perimeter of the collar 810. Shown at FIG. 8 is an angle at the dove's wingtip, defined by the exterior perimeter of the collar 810, and having vertex V2 pointing outward of the exterior perimeter. The beak may be considered another such angle. These angles are used simply as non-limiting examples.

For particularly fanciful designs such as the dove in flight of FIG. 8, a patient might choose to have the design highlighted or enhanced with tattoos, such as the lines separating feathers in the tail and wing of the FIG. 8 dove. Since the injection port is itself intended to be temporary, a dissolvable tattoo ink can be used so the tattoo can be eradicated by a simple injection soon after the port is surgically removed.

FIG. 9 is another embodiment in which there is a circular septum 910 outlined by a circular interior perimeter of the collar 910, but in which the exterior perimeter of the collar 910 is in the shape of a sunflower or a generic flower with extending petals. These also can be highlighted by tattoo as with FIG. 8 above, such as for example to make the tips of the petals more pointed, to add veins to the petals, to add a stem extending from the flower head, to add color to the design and/or to add scallops over the septum to mimic sunflower seeds. Where the outward facing petal points are well rounded as in FIG. 9 rather than more sharply defined as in FIGS. 3-4 the exterior perimeter may give the viewer the impression of a flower instead of a star, and exemplary embodiments of the invention can have such a flower defining any various number of distinct petals. By example and not by way of limitation the flower formed by the exterior perimeter of the FIG. 9 collar 910 defines twelve petals. At FIG. 9 there is shown by reference number one of the angles whose vertex V1 points inward of the exterior perimeter. The petal tips in other embodiments may also form angles whose vertex points outward of the exterior perimeter.

The above are non-limiting embodiments which are presented to give an understanding of the more general principles of the invention, some of which are set forth in the claims below. Some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

I claim:

1. An implantable surgical injection port comprising:
a housing defining a fluid reservoir;
a septum, at least a portion thereof being penetrable by a needle, disposed across an opening of the housing so as to enclose the fluid reservoir; and
a collar having an upper surface facing opposite the septum, the upper surface defining an exterior perimeter and an interior perimeter that circumscribes at least the needle penetrable portion of the septum;
in which at least one of the interior perimeter and the exterior perimeter defines a shape that is not elliptical.

2. The implantable surgical injection port of claim 1, in which the interior perimeter comprises at least one angle having a vertex facing inward of the interior perimeter.

3. The implantable surgical injection port of claim 2, in which the angle is acute.

4. The implantable surgical injection port of claim 3, in which the interior perimeter comprises at least three acute angles having respective vertexes facing inward of the interior perimeter.

5. The implantable surgical injection port of claim 1, in which the exterior perimeter comprises at least one angle having a vertex facing outward of the interior perimeter.

6. The implantable surgical injection port of claim 5, in which the angle is acute.

7. The implantable surgical injection port of claim 6, in which the exterior perimeter comprises at least three acute angles having respective vertexes facing outward of the interior perimeter.

8. The implantable surgical injection port of claim 1, in which the exterior perimeter and the interior perimeter define the same shape.

9. The implantable surgical injection port of claim 1, in which the shape is one of a heart and a star, in which the star comprises between three and six points.

10. The implantable surgical injection port of claim 1, in which the exterior perimeter and the interior perimeter define different shapes.

11. The implantable surgical injection port of claim 1, in which the upper surface is configured to be nearest to a patient's skin when the surgical injection port is subcutaneously implanted in combination with a catheter.

12. The implantable surgical injection port of claim 1, in which the exterior perimeter defines an outline of an animal.

13. The implantable surgical injection port of claim 12, in which the interior perimeter defines an elliptical shape.

14. The implantable surgical injection port of claim 12, in which the outline of an animal is an outline of a bird.

15. The implantable surgical injection port of claim 1, in which the said collar is a secondary solar, the implantable surgical injection port further comprising a primary collar which affixes the septum to the housing, and in which the secondary collar is coupled to the primary collar.

16. The implantable surgical injection port of claim 15, in which the secondary collar is removably coupled to the primary collar via mating surfaces which each define an elliptical perimeter.

17. The implantable surgical injection port of claim 16, in which the elliptical perimeters are circular.

18. The implantable surgical injection port of claim 1, in which the interior perimeter defines an ellipse and the exterior perimeter defines an outline of a flower.
